(19) United States
(12) Patent Application Publication
(10) Pub. No.: US 2010/0184220 A1
(43) Pub. Date: Jul. 22, 2010

(54) PREVASCULARIZED TISSUE TRANSPLANT CONSTRUCTS FOR THE RECONSTRUCTION OF A HUMAN OR ANIMAL ORGAN

(75) Inventors: Gouya Ram-Liebig, Dresden (DE); Manfred Wirth, Dresden (DE)

Correspondence Address: Emerson, Thomson & Bennett, LLC, 777 W. Market Street, Akron, OH, 44303, US

(73) Assignee: THE TECHNISCHE UNIVERSITAT DRESDEN, Dresden (DE)

(21) Appl. No.: 12/280,916

(22) PCT Filed: Feb. 28, 2007
(86) PCT No.: PCT/DE07/00368

§ 371 Date: Jan. 12, 2009

(30) Foreign Application Priority Data
Feb. 28, 2006 (DE) 10 2006 009 539.1

(51) Int. Cl. C12N 5/071

(52) U.S. Cl. 435/395

(57) ABSTRACT

The invention relates to a tissue transplant construct for the reconstruction of a human or animal organ. Here said tissue transplant construct is intended to comprise

(a) a biocompatible acellular membrane and
(b) microvascular endothelial cells which penetrate the membrane,
wherein microvascular structures of microvascular endothelial cells are formed within the membrane.

The present invention relates to tissue transplant constructs for the reconstruction of a human or animal organ, a process for the preparation of such a tissue transplant construct, as well as uses of the tissue transplant construct. Particularly, the present invention relates to a tissue transplant construct for the reconstruction of the lower urological organs and particularly the urinary bladder.

The aim of the tissue architecture (“tissue engineering”) is to replace affected, injured, or missing body tissue by biologically acceptable substitutes. At present there are investigated both the seeded technique and the unseeded technique in the tissue architecture for acceleration of the tissue regeneration (Alberti et al., 2004). The seeded technique (or cellular tissue architecture) uses biologically degradable membranes, which were coated with primarily cultivated cells in vitro, wherein the cells have been obtained from a biopsy of native host tissue. This assembled transplant is then introduced into the host to continue the regenerative process. The unseeded technique comprises the direct placement in vivo into the host of an uncoated biologically degradable material, which then is intended to function as a scaffold causing the natural regenerative process in vivo to take place. These techniques promote a tissue regeneration that is similar to the normal embryonic development of the organs of interest.

Acellular biological materials exhibit good results in generating tissue in vivo as seeded and unseeded membranes (Atala et al., 2000). Indeed, these biologically degradable materials in the tissue architecture of different organs have been used as natural, degradable and porous material and are known to provide biologically specific signals for the molecular interaction with the cultivated cells in vitro and more over to interact with the cells of the target tissue after implantation.

However, once the transplant has been implanted into the host, the capability for keeping alive the cells grown on the surface or within the matrix in vitro (seeded technique) or these that infiltrate the matrix after implantation (unseeded technique) is a critical obstacle. It has been shown, that one portion of the tissue having a volume exceeding a few cubic millimeters can not survive by diffusion of nutrients but requires the presence of blood capillaries for supplying essential nutrients and oxygen (Mooney et al., 1999). Hence the released vascularization may cause the failure of an implant. Up to now the success of the implantation techniques was limited to relatively thin or avascular structures (for example skin and cartilage), where the vascularization after implantation through the host is sufficient to fulfill the requirements of an implant for oxygen and nutrients (Jam et al., 2005). The vascularization is still a critical obstacle for the development of thicker, metabolically demanding organs such as heart, brain, and urinary bladder.

The capability for prevascularizing tissue scaffolds is a fundamental therapeutic strategy and a significant step in the tissue technique by avoiding limited tissue regeneration. A prevascularized matrix accelerates its vascularization and improves its blood circulation and its survival in vivo. Particularly complex tissues and organs require a vascular supply to ensure survival of the transplant and to make bioartificial organs functioning (Mertsching et al., 2005). One way for neovascularization is the formation of novel vessels from endothelial cells. This method, referred to as vasculogenesis (angiogenesis), typically takes place in the course of the embryonic development during the formation of organs (Risau et al., 1995).

Most important is the prevascularization of materials for the reconstruction of the lower urinary passages (urinary bladder, ureter and urethra). These organs exhibit a rich supply via blood vessels. In the seeded technique of the tissue architecture of the lower urinary organs the trials so far only were limited to cultivation of urothelial cells and muscle cells (Alberti et al., 2004) on biological membranes. These cells can easily be harvested from small bladder biopsies.

At present it is not fully understood what type of endothelial cells can be used for the prevascularization of biological scaffolds intended for implantation in vivo. It is known, that the phenotypes of endothelial cells vary strongly depending on the type of the vessels and the organ and these tissue specificity plays an important role in the local physiology of the organs (Jam et al., 2005).

Schultheiβ et al. describe in “Biological vascularized matrix for bladder tissue engineering: matrix preparation, reseeding technique and short-term implantation in a porcine model”, J. Urol. January 2005; 173(1): 276-80, a biological acellular matrix that is colonized with smooth muscle cells, urothelial cells, and endothelial precursor cells. The precursor cells were recovered from blood cell fractions. For the manufacture of a tissue transplant construct the matrix is seeded in addition to smooth muscle cells and urothelial cells with endothelial precursor cells from the blood. However, these cells are not able to form vascular structures in the matrix. Moreover the endothelial precursor cells are not organ-specific. In addition they are not mature endothelial cells.

The object of the invention is to eliminate the disadvantages according to the prior art. More particularly, there is given a tissue transplant construct, which in the case of coating the membrane with endothelial cells (and the formation of vascular structures inside the membrane) allows a fast and better generation of the surrounding tissues in the scaffold after implantation or which in the case of coating the membrane with endothelial cells as well as further organ-specific cells (such as urothelial cells, muscle cells and/or stromal cells) allows a better supply of the cells contained therein after implantation.

This aim is solved by the characteristics of claims 1, 11, and 28. Suitable embodiments of the invention result from the characteristics of the pendent claims.

In accordance with the invention there is intended a tissue transplant construct for the reconstruction of a human or animal organ, comprising

(a) a biocompatible acellular membrane and
(b) microvascular endothelial cells which penetrate the membrane,
wherein microvascular structures of microvascular endothelial cells are formed inside the membrane.

The microvascular endothelial cells are preferably organ-specific microvascular endothelial cells.

The microvascular structures formed from the applied microvascular endothelial cells allow a supply with nutrients and oxygen of the remaining cells of the lower urinary organs, in particular urothelial cells, muscle cells, stromal cells, after implantation of the construct. Additionally other cells are supplied which penetrate into the construct after implantation.

The tissue transplant construct according to the invention may comprise further tissue-specific cells such as urothelial cells and/or smooth muscle cells and/or stromal cells, which have been applied to and cultivated on the membrane before implantation. These cells are then also supplied by the microvascular structure after implantation of the tissue transplant construct.

Thus the invention provides for the first time a tissue transplant construct, which allows a supply of cells not only on its surface but also inside the construct. Therefore the constructs are well suited for the reconstruction of organs.

In particular, the invention employs organ-specific microvascular endothelial cells of the urinary bladder (microvascular bladder-endothelial cells) for the prevascularization of matrices intended for use to reconstruct the lower urinary organs. The microvascular bladder-endothelial cells could be isolated from a small biopsy material from this organ and used to form endothelial vascular networks in different biological acellular matrices. Preferred matrices are acellular bladder matrix, acellular urethra matrix, acellular bladder submucosa, small intestine submucosa, acellular skin matrix, acellular aorta matrix.

Alternatively to microvascular bladder-endothelial cells there may also be used dermal microvascular endothelial cells. Dermal microvascular endothelial cells may also be used for the preparation of a tissue transplant construct that can be employed both for reconstruction of the lower urinary organs and also for other organs. However, for the reconstruction of the urinary bladder the use of microvascular bladder-endothelial cells is preferred due to the tissue specificity.

Particularly the invention enables the cultivation of microvascular endothelial cells on different biological matrices in vivo and the formation of a stable vascular network construction for the reconstruction of soft tissue in particular of the urinary bladder, ureter and urethra. The cultivation takes place in culture systems providing angiogenic growth factors to induce prevascularization of matrices with microvascular bladder-endothelial cells. The formation of vessel structures in the finished tissue transplant constructs is induced by bladder stromal cells, marrow stromal progenitor cells and bladder urothelial cells, in particular by a medium conditioned with these cells. The stromal cells, marrow stromal progenitor cells, and epithelial cells such as urothelial cells are known to be associated with the induction of the vasoformation in vitro (Velazquez et al., 2002; Markowicz et al., 2005; Thompson et al., 2006).

However, the invention is not limited to a tissue transplant construct for the reconstruction of the lower ureter but may also be adapted to other organs, in particular soft part organs.

The term “organ-specific” in this connection is intended to be understood this way that cells of the same or of an identical organ to be reconstructed are applied to the membrane. If, for example, the intention is to reconstruct an urinary bladder, by organ-specific microvascular endothelial cells in this connection microvascular endothelial cells from the urinary bladder are meant.

Organs are functional units of the body. The preferred example is the urinary bladder.

The term “microvascular structure” means the formation of a capillary structure inside the membrane. A capillary structure preferably consists of cord-like and/or tubular units formed from microvascular endothelial cells. The tubular units preferably have completely developed lumina. The cord-like and/or tubular units are preferably cross-linked. After implantation of the construct according to the invention into the organ to be reconstructed and the connection of the microvascular vessel structure of the construct with the in vivo vessel structure there is ensured the supply of the endothelial cells and other cells of the construct. Thus the construct can be supplied with nutrients and oxygen via the microvascular structures immediately after implantation. The process of the vasoformation is also referred to as vascularization. By prevascularization is meant that the microvascular structure is already present before the implantation.

The terms “membrane” and “matrix” are used synonymously herein unless indicated otherwise. The membrane ought to comprise the components of the extracellular matrix (ECM), especially the growth factors thereof. It is preferred that the membrane is a biological membrane. The membrane constitutes the three dimensional biological scaffold into which the applied microvascular endothelial cells penetrate in the course of cultivation.

The membrane is preferably an acellular human or porcine urinary bladder, an acellular human or porcine urinary bladder submucosa, an acellular human or porcine dermis, an acellular human or porcine small intestine, an acellular human or porcine aorta, or an acellular human or porcine urethra. The membrane contains collagen. Suitably the membrane is mechanical stable.

Preferably the outer surface of the membrane is up to 40 cm² but may also be larger. The thickness of the membrane is preferably in the range of from 100 μm to 100 mm.

By reconstruction is meant the replacement of damaged or diseased regions of a human or animal organ, in particular of an urinary bladder, an urethra, or an ureter.

By the term “penetration” is to be understood herein that microvascular endothelial cells penetrate from the surface of the membrane into its inside and that the inside of the membrane is colonized by the microvascular endothelial cells. Herein the term “penetration” is intended to correspond to the term “invasion”.

By stromal induction is to be understood the proliferation of the microvascular endothelial cells induced by stroma. Preferably the stromal induction is an induction via bladder stromal cells and marrow stromal progenitor cells.

By epithelial induction is to be understood the proliferation of the microvascular endothelial cells induced by epithelial cells. In the case of the lower urinary pathways the epithelial cells are urothelial cells so that then can be spoken of urothelial induction.

By epithelial-stromal induction is to be understood the proliferation of the microvascular endothelial cells that is induced by a mixture of stromal cells and epithelial cells. Preferably the urothelial-stromal induction is an induction via urothelial bladder stromal cells. In the case of the lower urinary pathways the epithelial cells are urothelial cells so that then can be spoken of an urothelial-stromal induction.

Hereinafter a mixture of bladder stromal cells and bladder urothelial cells is also referred to as urothelial bladder stromal cells, urothelial stromal cells, stromal urothelial cells, or bladder urothelial stromal cells.

An urinary bladder consists of mucosa (urothelial cells) and stroma (fibroblasts, smooth muscle cells and endothelial cells).

The tissue transplant construct is preferably used for the reconstruction of the lower urinary pathways, in particular of the urinary bladder, the urethra, or the ureter. In this case the human or animal organ to be reconstructed is an urinary bladder, whereas the organ-specific microvascular endothelial cells are microvascular bladder endothelial cells and/or dermal microvascular endothelial cells.

More preferred the microvascular endothelial cells are autologous microvascular endothelial cells, i.e. the microvascular endothelial cells are isolated from the tissue of a patient whose organ is to be reconstructed with the tissue transplant construct according to the invention.

Also preferred the microvascular endothelial cells may be isolated from the skin and used for the prevascularization of the membrane.

According to the invention there is further provided a method for the preparation of the tissue transplant construct for the reconstruction of a human or animal organ, comprising the steps of

(a) isolating microvascular endothelial cells;
(b) applying the microvascular endothelial cells onto a biocompatible acellular membrane, and
(c) cultivating the microvascular endothelial cells, which have been applied onto the biocompatible acellular membrane, under stromal induction or under epithelial-stromal induction.

The stromal induction is preferably conducted using human or animal bladder stromal cells or human or animal marrow stromal progenitor cells.

Cultivating under stromal induction is preferably conducted by means of a conditioned medium, wherein the conditioned medium has been conditioned by means of human or animal bladder stromal cells, or human or animal marrow stromal progenitor cells. A conditioned medium may be obtained by cultivating an unconditioned medium with human or animal bladder stromal cells or human or animal marrow stromal progenitor cells and subsequently removing the medium as a supernatant from the bladder stromal cells or the marrow stromal progenitor cells. The conditioned medium is hereinafter also referred to as stroma-conditioned medium or stromal cells-conditioned medium.

Preferably the epithelial-stromal induction is an urothelial-stromal induction that is conducted using urothelial bladder stromal cells.

Especially preferred cultivation under epithelial-stromal induction is conducted by means of a conditioned medium, wherein the conditioned medium has been conditioned by means of human or animal epithelial cells, in particular urothelial cells and bladder stromal cells. A conditioned medium may be obtained by cultivating an unconditioned medium with human or animal epithelial cells and subsequently removing the medium as a supernatant from the mixture of epithelial cells and stromal cells.

Especially preferred cultivation under epithelial induction is conducted by means of a conditioned medium, wherein the conditioned medium has been conditioned by means of human or animal epithelial cells, in particular urothelial cells. A conditioned medium may be obtained by cultivating an unconditioned medium with human or animal epithelial cells and subsequently removing the medium as a supernatant from the epithelial cells.

Preferably the microvascular endothelial cells are extracted from the stromal tissue of the organ to be reconstructed. This may in one embodiment of the invention take place by

(i) digesting the stromal tissue by means of a collagenase;
(ii) separating the microvascular endothelial cells from the mixture obtained in step (i) using paramagnetic lectine- or antibody-coupled particles, wherein the antibodies are monoclonal antibodies for the platelet-endothelial cell adhesion molecule 1 (PECAM 1) or CD105;
(iii) propagating the thus obtained microvascular endothelial cells, and
(iv) separating the microvascular endothelial cells from the mixture obtained in step (iii) using paramagnetic lectine- or antibody-coupled particles, wherein the antibodies are monoclonal antibodies for the platelet-endothelial cell adhesion molecule 1 (PECAM 1) or CD105, and wherein the thus obtained microvascular endothelial cells are a mixture with a purity of at least 95% based on the number of all separated cells.

Especially preferred the residue of the mixture remained in step (ii) is used for the preparation of the conditioned medium. Alternatively, following step (c) the residue of the mixture on the prevascularized membrane may be cultivated with urothelial cells and thus form a complex vital tissue transplant construct.

The invention offers the possibility to reconstruct the lower urinary pathways with the inventive prevascularized biological matrices at which the tissue generation in vitro is started, wherein simultaneously the proliferation of microvascular bladder endothelial cells is accelerated and the microvessel formation thereof is improved. The tissue transplant construct according to the invention in addition to the microvascular endothelial cells may contain further tissue-specific cells like epithelial cells, and stromal cells.

The advantageous results of the inventions are based on the following findings of the inventors: The angiogenesis is a complex process involving an extracellular matrix (ECM) and vascular endothelial cells and is regulated by different angiogenic factors. The capability of forming a capillary/tubular network is a special function of endothelial cells (EC) and requires a specific cascade of processes consisting of endothelial cell invasion, migration, proliferation, formation of tubes, and anastomosis between the structures (Cameliet et al., 2000; Yancopoulos et al., 2000; Blau et al., 2001; Ferrara et al., 1999; Keshet et al., 1999). In this way endothelial cells are guided by the signals from the microenvironment of the matrix surrounding them (Berthod et al., 2006).

To promote the microvessel formation and its maintaining in vitro an acellular urinary bladder or ureter matrix is preferably used, comprising the main components of bladder and ureter interstitium as physiological matrix for the endothelial cell invasion and differentiation respectively. Also preferred is the use of other biological acellular matrices, comprising collagen as main component, which is also the main component of the interstitium. The fact that the formation of microvascular structure inside all of the matrices was observed shows that the capability of human microvascular endothelial cells is not limited to only one type of the biological matrix.

Endothelial cells are also known to be activated by signals from other cells of their microenvironment (Berthod et al., 2006; Velasquez et al., 2002). Stromal cells (fibroblasts and smooth muscle cells) are often attached to endothelial cells contributing to morphogenesis and final differentiation to the capillary/tubular phenotype. Stromal cells, and in particular fibroblasts, are associated with the production of matrix proteins serving as scaffold for vasculature and other organ structures. These cells are also a rich source for angiogenic growth factors (Honorati et al., 2005) for guiding the proliferation and differentiation of endothelial cells (Erdag et al., 2004; Hudon et al. 2003) and associating with the stabilization of vessels in vitro (Velazquez et al., 2002). It is further known that epithelial cells secrete growth factors that induce the vascularization (Thompson et al., 2006).

Darland et al., 2001 have observed that stromal cells like differentiated pericytes in culture with endothelial cells generate growth factors that promote survival and/or stabilization of endothelial cells.

Adult marrow mesenchyme stem cells are multipotent and strongly proliferating cells that can release different growth factors (Tang et al., 2004), which promote survival and/or stabilization of endothelial cells (Markowicz et al., 2005). These cells are known to provide a local environment that promotes ingrowth of vessels in a damaged location (Gruber et al., 2005).

In the present invention the induction effect of bladder stromal cells, marrow stromal progenitor cells, and bladder urothelial cells through a conditioning medium has been studied (Velazquez et al., 2002; Gruber et al., 2005; Thomson et al., 2006). For this, supernatants of stromal cells originating from the marrow, organ-specific bladder stromal cells, and organ-specific bladder urothelial cells were used to accelerate the proliferation of isolated microvascular bladder endothelial cells and dermal microvascular endothelial cells attached to the biological scaffold by means of the paracrine function of the stromal cells and urothelial cells.

In experiments taken as a basis for the invention there were first cultivated human microvascular bladder endothelial cells or dermal microvascular endothelial cells as a monolayer on the matrices. Then the cultivated cells were fed with stroma-conditioned or urothelial-stromal-conditioned medium. In control experiments the cultivated cells were fed with urothelial-conditioned medium or unconditioned medium. To determine the capability of stromal cells and urothelial cells to modulate the proliferation, differentiation, and stabilization of microvascular endothelial cells on acellular biological degradable materials there was evaluated the angiogenesis in vitro by histological and immunohistochemical analyses for determining the proliferation, penetration, formation of a capillary/tubular network and the phenotype. There are three angiogenic parameters that have been suggested in the literature for the evaluation of the formation of a tubular network: capillary length, number of capillaries, and relative capillary region. In this experiments performed the capillary length in an assay was determined (Watanable et al., 2005). Here the length of the capillaries was determined each in three histological longitudinal sections of three different constructs of the cells extracted each from one urinary bladder in light microscope analyses.

The histological and immunohistochemical studies of microvascular endothelial cells on three dimensional biological matrices conditioned with stromal cells and mixtures of stromal and urothelial cells have shown activated invasive cells that degraded their pericellular matrix whereas simultaneously their differenciated phenotype was maintained. Once these cells were released into the three dimensional extracelluar matrix they began migrating to other cells and oriented to cords of endothelial cells. The micro tube formations grew toward each other and formed anastomoses with other micro tubes under paracrine cytokine signals of stromal cells and mixtures of stromal and urothelial cells. Compared with this system the cultures fed with unconditioned media or only urothelial-conditioned medium exhibited only a small increase of the number of endothelial structures, a smaller surface covered by endothelial cells, and a lower percentage of angioid structures with lumen in comparison to stroma-conditioned cultures and urothelial-stromal-conditioned cultures.

The proliferation assay with KI-67 has shown that in unconditioned culture systems or in only urothelial-conditioned as well as in urothelial-conditioned culture systems the endothelial cells were latent in a period of one to two weeks, for the most part two weeks. In stromal- or urothelial-stromal-conditioned culture systems the endothelial cells stayed proliferating for at least four weeks giving constructs with a higher cell density as that of constructs in unconditioned or only urothelial-conditioned culture systems.

The assay for determining the formation of a capillary/tubular network has also shown longer capillary formations per field in the stromal- or urothelial-stromal-conditioned systems.

Therefore the stromal cells as well as mixtures of stromal and urothelial cells provide in their supernatant factors that can stimulate the invasion of endothelial cells and the formation of micro tube structures. These results suggest that stromal cells or mixtures of stromal and urothelial cells provide critical signals with mitogenic and motogenic endothelial effects promoting the construction of tubular structures from endothelial cells.

The development of capillary microvascular structures not only requires stromal cells or a mixture of stromal cells and urothelial cells, but also the presence of an extracellular matrix (ECM). In fact, endothelial cells do not form tubular structures under stromal induction or urothelial-stromal induction in two-dimensional conventional culture dishes. ECM components of the biological membranes can both function as a scaffold and as an inductor for endothelial cells. Obviously there are provided synergistically to the activation of the endothelial cells by exposing them to the extracellular matrix signals for accelerating the migration, the survival, and the differentiation of the endothelial cells for the real capillary morphogenesis by stromal cells or mixtures of stromal and urothelial cells.

The fact that an increased proliferation and differentiation of endothelial cells by media that have been conditioned by means of bladder stromal cells, marrow stromal progenitor cells, or a mixture of urothelial cells and stromal cells does not suggest to organ-specificity with regard to the effects on the capillary-like morphology mediated by the stromal cells or mixtures of stromal and urothelial cells, implicates a critical role of stromal cells or of the mixture of urothelial cells and stromal cells regarding the control of the behavior of the endothelial cell.

The vascular endothelial growth factor (VEGF) represents one of the most effective endothelial cell mitogens (Pepper et al., 1992-Lazarous et al., 1997). It is one of the factors that stimulate the expression of matrix metalloproteinasen, proliferation, migration, and formation of tubes of isolated endothelial cells in vitro and the development of vessels in vivo (Gruber et al., 2005).

To determine the presence of these vasculogenic and angiogenic key factors in the conditioned media the concentration of the vascular endothelial growth factor (VEGF) was studied in ELISA tests. The results have shown detectable concentrations of VEGF suggesting that measurable concentrations of this cytokine are secreted by stromal cells. The constructs according to the invention are thus specifically based on the capacity of the growth factor expression by stromal cells and mixtures of stromal cells and urothelial cells. With respect to these growth factors the urothelial cells produced higher values. Said cytokine has been developed strongest by the mixed cultures of urothelial cells and stromal cells, wherein the highest concentration of VEGF was measured in the medium conditioned with the mixture of stromal and urothelial cells.

The fact that the proliferation and the tubular-capillary formation by means of conditioned medium only with urothelial cells (despite of the production of VEGF by these cells) were hardly induced indicates that the additionally presence of the stromal cells is advantageous for the induction of the endothelial cells. Thus the inducing effect of stromal cells as far as they are concerned can be increased by urothelial cells.

On the other side after cultivation of the microvascular endothelial cells as monolayer on the acellular matrices the further cultivation without a conditioned medium (i.e. without stromal or urothelial-stromal induction) or only with urothelial-conditioned medium resulted in the detachment of cells from the monolayer and the invasion of these cells into the matrix, but the invasion into the underlying matrix was not that high such as under conditioned requirements with stromal cells or with a mixture of stromal and urothelial cells. Moreover the endothelial cells arranged only in a few polarized cords with low branching and only a few thereof had a fully developed lumen. Hence the acellular matrix is essential for the initial migration into the lower acellular layer but not sufficient for the promotion of the further locomotion and for the formation of mature tubular structures inside the acellular matrix. These results show that the stromal induction or the urothelial-stromal induction is important for the adequate migration and invasion, and for the formation of mature luminal structures.

Taken together, the results in vitro on which the invention is based show that the prevascularization of biological acellular matrices by means of isolated autologous microvascular endothelial cells harvested from a small bladder biopsy or skin and induced by bladder stromal cells, marrow stromal progenitor cells, or a mixture of bladder urothelial cells and bladder stromal cells represents a successful possibility for treating the deficiencies of the lower urinary pathways.

However these findings are not limited to the lower urinary pathways, in particular the urinary bladder, but may also be adapted to other organs, in particular soft part organs.

Hereinafter the invention is explained in more detail with the help of examples with respect to the drawings. The figures show the following:

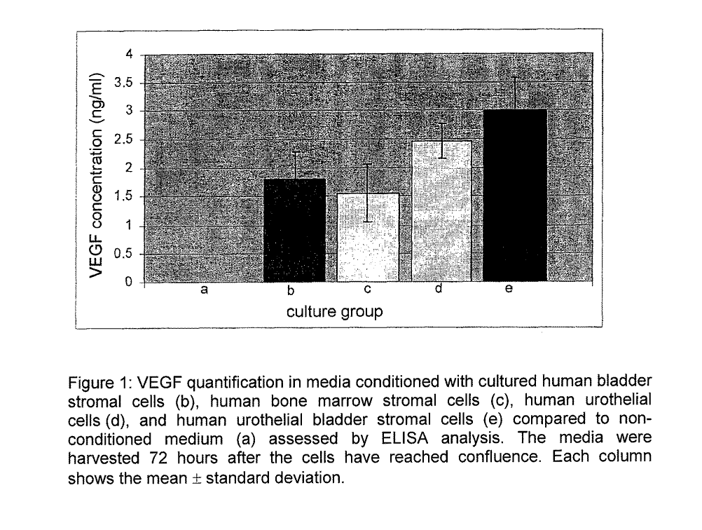

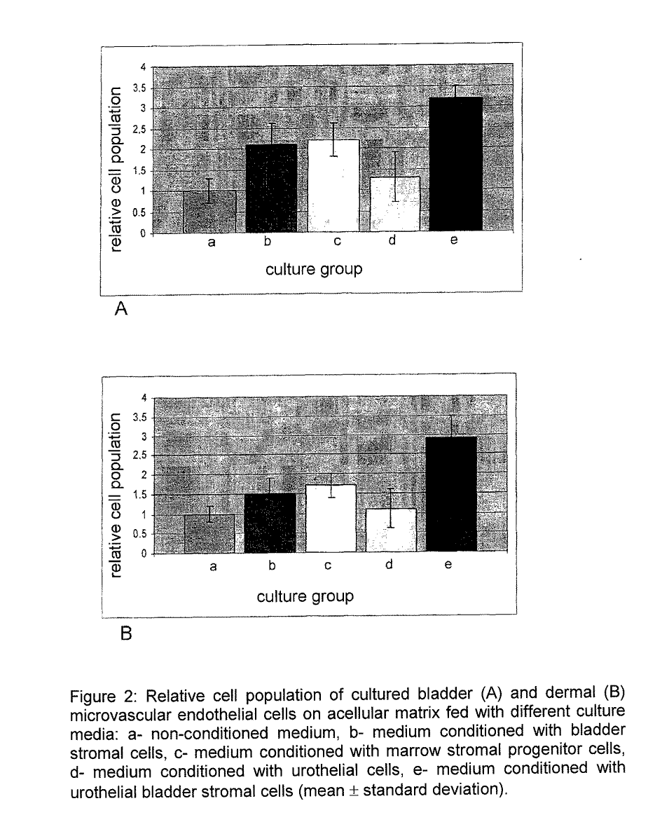

FIG. 1 a histogram showing the VEGF-concentration in conditioned and unconditioned media;

FIG. 2 a histogram showing the cell population of microvascular endothelial cells after cultivation with different conditioned media inside the membrane;

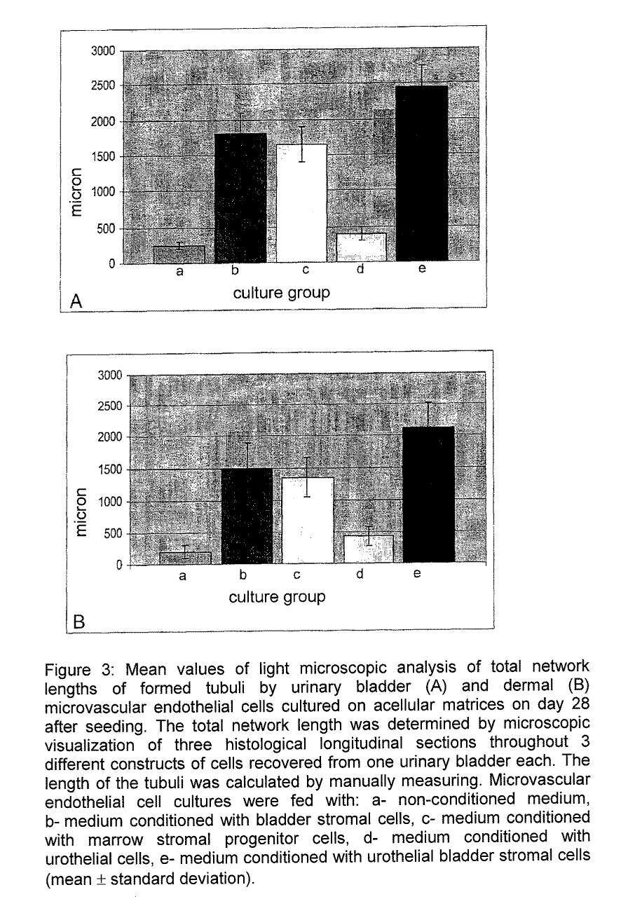

FIG. 3 a histogram showing the length of microvascular structures in tissue trans-plant constructs the microvascular endothelial cells of which have been cultivated with different culture media;

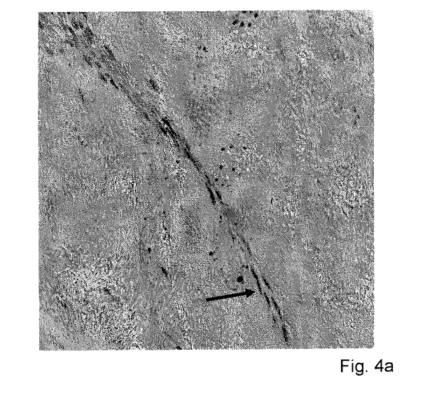

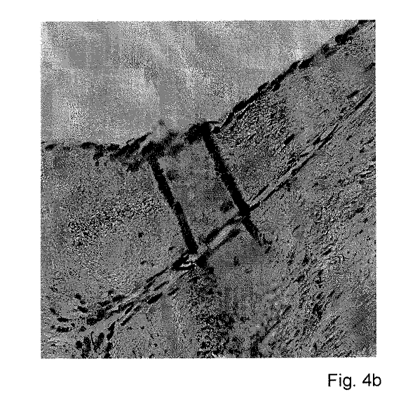

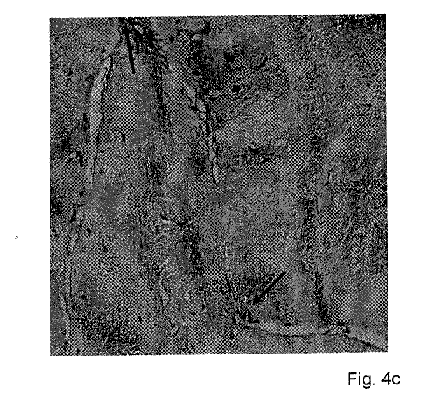

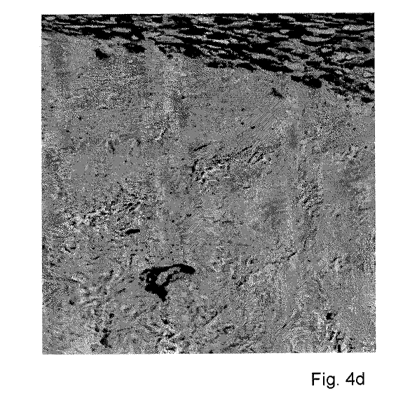

FIG. 4A a histological imaging for the characterization of an in-vitro-culture of seeded microvascular bladder endothelial cells on the surface of an acellular matrix under stromal and urothelial-stromal induction on day 14, respectively, wherein the bladder endothelial cells are detached from the surface of the matrix and penetrated into the scaffold of the matrix when inducing with stromal and urothelial-stromal soluble factors, respectively. First there is formed a cord lined with cells. Gradually, there can be seen a fully developed lumen (arrow);

FIG. 4B a histological imaging for the characterization of an in vitro culture of seeded microvascular bladder endothelial cells on the surface of an acellular matrix under stromal and urothelial-stromal induction on day 14, respectively, which have formed a tube at the inside of the scaffold under induction;

FIG. 4C a histological imaging for the characterization of an in vitro culture of seeded microvascular bladder endothelial cells on the surface of an acellular matrix under induction with a stromal and urothelial-stromal conditioned medium on day 21, respectively, wherein new branchings (arrows) of the tube with the fully developed lumen lined with microvascular bladder endothelial cells can be seen; and

FIG. 4D an immunohistochemical imaging for the characterization of an in vitro culture of seeded microvascular bladder endothelial cells on the surface of an acellular matrix under induction with a stromal and urothelial-stromal conditioned medium on day 28, respectively, wherein the luminal structure is lined with the bladder endothelial cells and wherein a positive expression of the endothelial marker von Willebrand factor can be seen. The multiple layer of seeded microvascular bladder endothelial cells on the surface of the matrix also expressed positive the said antibody.

### EXAMPLES

The following examples illustrate the inventive method for the preparation of tissue transplant constructs for the lower urinary organs.

Unless indicated otherwise the abbreviations used have the following meaning:

| | |
|---|---|
| b-FGF | b fibroblast growth factor |
| DMEM | Dulbecco's modified Eagle's Medium |
| UEA-1 | Ulex Europeaus Agglutinin 1 Lectin |

### Materials and Methods:

### (a) Preparation of Acellular Matrices

Human or porcine urinary bladders and bladder submucosa, urethra, dermis and small intestines were washed with phosphate buffered saline (PBA) (Invitrogen, Germany) and subjected a treatment with Triton X 1% for 24 to 48 hours under gentle shaking at 37° C. Acellularity of the matrices was controlled histologically.

### (b) Isolation, Culture and Characterization of Human Microvascular Bladder Endothelial Cells and Dermal Microvascular Endothelial Cells

For the isolation of microvascular bladder endothelial cells human bladder tissue was harvested from four patients undergoing an open bladder surgery. Urothelium was microdissected and the stromal tissue was chopped for enzyme dissociation and digested with 1 mg/ml collagenase Type II (Worthington Biochemical Corporation, USA)/Dispase (0.05 mg/ml) (Invitrogen)/16 μg/ml deoxyribonuclease Type I (Boeringer Mannheim GmbH, Germany) in DMEM medium containing 0.2% serum albumin (Sigma Aldrich, Germany) for 2 hours at 37° C. with continuous agitation. The digested material was centrifuged at 2000 rpm, and the cell suspension was collected, diluted in DMEM medium, and filtered through a 40 mm nylon mesh cell strainer (BD Labware, Germany). The filtered material was diluted in DMEM medium (Invitrogen, Germany) containing 2.5% human serum. Polystyrene paramagnetic beads (Dynabeads) (Dynal, Germany) were coupled with lectin UEA-1 (Sigma Aldrich, Germany) or with monoclonal antibody for platelet endothelial cell adhesion molecule-1 (PECAM 1; CD 31) (Dakocytomation, Denmark) by incubating 10⁷ Dynabeads with the lectin or the antibody CD 31 for 24 hours at room temperature, with overhead rotation, according to the manufacturer's instructions. The Dynabeads were collected using a device for concentrating the magnetic particles and washed three times in PBS/0.1% serum albumin. Bladder microvascular endothelial cells were counted and incubated at 4° C. for 10 min with the UEA 1 or CD 31-coated Dynabeads with overhead rotation. The positively selected cells were subsequently removed from the mixed cell population using a device for concentrating the magnetic particles (Dynabeads/endothelial cell ratio 10:1). The Dynabead-attached cells (endothelial cells) were recovered and washed ten times in DMEM/2.5% human serum. The negatively selected non-attached cells (bladder stromal cells) were washed in PBS and cultivated in culture flasks at the conditions mentioned under (c). The purified endothelial cells were resuspended in culture medium with 10⁴ cells/cm² seeded in culture dishes. The medium was changed every 2 to 3 days. The cells were passaged at 70% confluence. After the second passage, cells were again purified with lectin UEA-1 or CD 31-coupled Dynabeads. The endothelial phenotype of the cells was confirmed by immunohistochemical staining using von Willebrand factor and CD 34 antibodies (both Dako). The cells could also be separated using Dynabeads to which CD105 and CD31 (Miltenyi, Germany), respectively was coupled.

The thus obtained microvascular bladder endothelial cells of the third to fifth passage were stored in liquid nitrogen and used for the experiments.

For the isolation of human dermal microvascular endothelial cells human foreskin of patients undergoing circumcision was washed many times with PBS. The dermis was separated from subcutaneous fat mechanically by micro scissors. For the separation of dermis from epidermis the tissue was incubated in 0.25% Dispase (Boeringer Mannheim, Germany) for 2 hours. The dermis was then chopped and incubated for 30 min in Trypsin (0.05%)/EDTA (0.01%) (Invitrogen, Germany). The digested cell suspension was filtered through a nylon mesh (40 μm pore size), and centrifuged. The cell pellets were diluted in EGM-2 (Cambrex, Germany) until subconfluence. The cells were subsequently taken from the culture dishes and the dermal microvascular endothelial cells were positively selected using Dynabeads coupled with CD-31. The cells of the third passage were subjected to a second separation procedure. The endothelial phenotype of the cells was confirmed by immunohistochemical staining for von Willebrand factor and CD34. The cells of the third to fifth passage were stored in liquid nitrogen and used for further experiments.

### (c) Isolation and Cultivation of Human Adult Bladder Stromal Cells and Marrow Stromal Progenitor Cells

The remaining (negatively selected) bladder stromal cells, which were not coupled to the Dynabeads, were washed in PBS and subsequently cultivated in DMEM (Invitrogen, Germany) with additional serum and penicillin-streptomycin. Immunohistochemical characterization was assessed with antibodies against α-smooth muscle actin, vimentin, and pancytokeratin.

Marrow stromal progenitor cells were harvested from marrow material obtained from iliac crest bone of six healthy adult patients by needle aspiration and collected in a heparinized 50 ml test tube. Aspirated material was mixed with an equal volume of DMEM/10% serum. The cell suspension was layered on top of 10 ml of Ficoll-Paque (Amersham Pharmacia Biotech AB, Sweden) and centrifuged at 400 g for 35 min at 4° C. Mononuclear cells were collected from interphase, filtered through a 100 μm nylon mesh cell strainer (Becton Dickinson; Germany) and resuspended in DMEM supplemented with human serum and penicillin/streptomycin at a concentration of 10⁶ cells/cm². Cells were characterized by flow cytometry. For this purpose marrow stromal progenitor cells were trypsinized, washed with PBS, and incubated with antibodies against CD34, CD45, CD44, CD73, CD90 (all Becton-Dickinson), and CD133 (Miltenyi Biotech; Germany). Analysis was performed with a FACScalibur flow cytometer (Becton Dickinson). Cells were expanded to confluence with changing the culture medium every 3 to 4 days.

Bladder stromal cells and marrow stromal progenitor cells of the first five passages were stored in liquid nitrogen and used for further experiments.

### (d) Isolation and Cultivation of Urothelial Cells and Urothelial Bladder Stromal Cells

For cultivation of bladder urothelial cells bladder mucosa obtained by means of micro-dissection from bladder biopsies was cut into thin pieces, digested in collagenase Type II 1 mg/ml for 2 hours, centrifuged at 2000 rpm for 5 min, and cultivated in 25 ml culture dishes with keratinocyte free serum (Cambrex).

To obtain a mixture of bladder urothelial cells and bladder stromal cells bladder biopsies obtained from bladder urothelium and bladder stroma were cut into small pieces, and digested and collected as described above. Cells of the first five passages were stored in liquid nitrogen and used for further experiments.

(e) Preparation of Media Conditioned with Bladder Stromal Cells, Marrow Stromal Progenitor Cells, Urothelial Cells, and Urothelial Bladder Stromal Cells:

Bladder stromal cells, marrow stromal progenitor cells, urothelial cells, and urothelial bladder stromal cells were each propagated independently of each other until confluence before the medium was changed to 20 ml of DMEM supplemented with serum and antibiotics for 72 h. The conditioned media were subjected to a sterile filtration. Each conditioned medium was supplemented with 20 ng/ml of b-FGF. Then the media were partitioned in aliquots and stored at −80° C.

ELISA was used to determine the concentration of the vascular endothelial growth factor (VEGF) in the conditioned media. For measuring the cytokine concentration a quantitative ELISA using commercial kits for the vascular endothelial growth factor was developed (R&D-Systems). For these tests the conditioned media were collected after 72 hours cultivation, centrifuged at 2000 rpm for 10 minutes, and passed through a 0.3 μm filter. All the assays were performed in triplicate on microtitre plates.

### (f) Cultivation of Microvascular Bladder Endothelial Cells and Dermal Microvascular Endothelial Cells on Biological Scaffolds

Microvascular bladder endothelial cells or dermal microvascular endothelial cells recovered in accordance to (a) were cultivated in culture dishes with trypsin/EDTA and taken at 70% confluence, counted, and centrifuged to obtain pellets of the desired number of cells. Cells were resuspended in culture medium and distributed homogeneously on the upper surface of the matrices at a final concentration of 10⁴/cm². Cell culturing was carried out for 28 days at 37° C. Cells were cultured with
(a) DMEM supplemented with serum and b-FGF (control group a); or
(b) bladder stromal-conditioned medium supplemented with b-FGF (culture group b); or
(c) marrow stromal progenitor cell-conditioned medium supplemented with b-FGF (culture group c); or
(d) urothelial-conditioned medium supplemented with b-FGF (culture group d); or
(e) urothelial bladder stromal-conditioned medium supplemented with b-FGF (culture group e).

All experiments were performed at least in duplicate and repeated for three times independently of each other.

### (g) Microscopic Analyses of Cultured Microvascular Bladder Endothelial Cells or Dermal Microvascular Endothelial Cells on Biological Scaffolds

At the end of each experiment formalin fixed biological scaffolds were dehydrated in series of increasing concentrations of alcohol, embedded in paraffin, and cut into sections of 6 mm width. The composition of the constructs was analyzed with heamatoxylineosin (HE) and immunohistochemistry using anti-human von Willebrand factor, endothelial cell specific lectin UEA-1, anti-human CD34, and anti-human CD31 (PECAM-1) and assessed with a phase-contrast microscope (Zeiss, Germany). Cell proliferation was assessed with the proliferation marker KI67. There are three parameters that can be used to assess the angiogenesis: capillary length, number of capillaries, and relative capillary area. We evaluated the capillary length in three different longitudinal sections of each specimen with the assay (Watanable et al., 2005). Here, the length of the capillaries was determined in each three histological longitudinal sections at three different constructs of cells obtained from each one urinary bladder by light microscope analyses. For this purpose, the length of capillary/tubular structures in histological sections within the matrix was quantified by manually measurement of the tubes. Cell densities of representative histological sections were quantified in the same way.

### Results

### Isolation and Culture of Human Bladder Microvascular Endothelial Cells and Dermal Microvascular Endothelial Cells

First there had to be evolved a selection process for isolation and preparation of primary cultures of microvascular bladder endothelial cells from bladder stromal tissue consisting mainly of smooth muscle cells, fibroblasts, and endothelial cells (see the above-mentioned section (a)). For that, lectin-coated or antibody-coated Dynabead particles were used. It was found, that an additional Dynabead purification was required during the first five passages to produce cultures of microvascular bladder endothelial cells with a purity of >95%. The attached Dynabeads were washed out within the first two passages and did not interfere with the microvascular endothelial cell growth or survival. The separation of the dermal microvascular endothelial cells resulted in a culture containing 80% of microvascular endothelial cells. A second separation of the third passage resulted in over 90% purity. The morphology of primary microvascular endothelial cells of the first passage showed some variability in size and shape. With increasing passage number, the cell population showed a homologous morphology. Cultured human microvascular bladder endothelial cells displayed the characteristic features of endothelial cells. They expressed factor VIII-related antigen and CD 34.

### Isolation, Culture and Characterization of Bladder Stromal Cells, Marrow Stromal Progenitor Cells, and Bladder Urothelial Stromal Cells.

Primary cultures of bladder stromal cells and marrow stromal progenitor cells were successfully established from bladder stroma and from aspirated marrow. A mixed culture of bladder urothelial cells and bladder stromal cells was also successfully and contained urothelial cells, smooth muscle cells, and fibroblasts. Pure urothelial cells were successfully recovered from urinary bladder mucosa. During the first 2 days isolated cells began to adhere and grow. They remained inactive for 3 to 5 days; then they began to propagate rapidly. Cultures of bladder stromal cells showed a uniform morphology with spindle shaped cells. In the primary culture)(P⁰) marrow stromal progenitor cells showed some variability in size and shape, consisting of three different morphologies. After the first passage these cells showed a homogenous, spindle shaped morphology. Bladder stromal cells and marrow stromal progenitor cells showed proliferative potentials and growth patterns similar to each other. In both cultures confluence was obtained after approximately 10 days. In the last passages (>P⁵) the spindle shaped cells began to display a broadened, flat morphology. Therefore, conditioned media were harvested only on cells of the passages 1 to 5 (P¹ to P⁵).

The cultured urothelial cells showed their typical epithelial morphology, which was uniformly under serum free conditioning. The mixed culture of urothelial cells and bladder stromal cells showed simultaneously cells with an urothelial phenotype and a stromal spindle shaped phenotype. Urothelial cell and urothelial bladder stromal cell cultures were passaged at sub-confluence. Here, also the cells of the first five passages were used for the preparation of conditioned media.

Marrow stromal progenitor cells were tested with flow cytometry for the presence or absence of characteristic haematopoietic markers. They typically expressed the antigens CD105 and CD73. Furthermore, they expressed the cells CD90 and CD44. They were negative for typical lymphocytic marker CD45 and the early haematopoietic markers CD34 and CD133.

Immunohistochemical analyses of bladder stromal cells showed that these cells consisted of two cell populations with about 50 to 60% of cells showing the positive expression for α-smooth muscle actin and 40 to 50% vimentin-positive cells without α-smooth muscle actin expression. The urothelial cell cultures formed a cell population, which solely expressed pancytokeratin. The urothelial bladder stromal mixed culture consisted of three cell populations at the same time, namely pancytokeratin-positive cells, α-smooth muscle actin-positive cells, and vimentin-positive cells. The latter two types of cells did not stain with pancytokeratin-antibody.

### ELISA Test

Media obtained from the cultured bladder stromal cells, marrow stromal progenitor cells, urothelial cells, or a mixture of bladder urothelial cells and bladder stromal cells showed detectable concentrations of VEGF in an ELISA test, wherein the highest concentration was calculated from the mixed culture of bladder urothelial cells and bladder stromal cells (FIG. 1).

### Microscopic Analyses of Cultured Microvascular Bladder Endothelial Cells on Biological Scaffolds

Microscopic examination of cultured microvascular endothelial cells on biological scaffolds showed the microvascular endothelial cell adhesion and survival on the biological scaffolds. After 24 h the microvascular endothelial cells had begun to migrate on the surface of the acellular membranes, while keeping their differentiated phenotype (positive immunoreactivity with von Willebrand factor, CD31, and CD34 and binding of UEA-1). They covered up to 45 cm² of the matrix surfaces and reached confluence as monolayer within two weeks, showing a higher migration capacity in the conditioned systems (culture groups b, c, and e). They adopted two different morphologies with a round phenotype and an elongated, tubular phenotype in the conditioned media (culture groups b, c, and e) and often round morphology in non-conditioned systems (control group a) and in only with urothelial cells conditioned systems (control group d). Cultures fed with the conditioned stromal media (control groups b, c, and e) showed a higher overall cell density after 28 days in culture, compared with the non-conditioned control scaffolds (control group a). The highest overall cell density was observed in the culture systems that have been conditioned with urothelial bladder stromal cells (culture group e). The medium conditioned only with urothelial cells (control group d) did not show an elevated cell number, compared with the non-conditioned systems. In fact in conditioned culture systems (culture groups b, c, and e) the microvascular endothelial cell proliferation in the matrix was observed up to 28 days as is to be seen by staining for the proliferation marker Ki67 and the cell density increased in course of time. In comparison thereto, microvascular endothelial cells in non-conditioned culture systems and in the medium conditioned with urothelial cells only (culture group d) kept proliferating in the first 7 to 14 days only, but died slowly over the last two to three weeks of the culture. The observed culture time was 28 days for all groups.

No significant distinctions were found between the bladder stromal cell-conditioned medium (control group a) and the marrow stromal progenitor cell-conditioned medium (culture group b). In these two culture systems the number of microvascular bladder endothelial cells was 2.1 to 2.2fold higher than in the control group a (FIG. 2). The number of dermal microvascular endothelial cells in these two culture systems (culture groups b and c) was 1.5 to 1.7fold higher than in the control group a (FIG. 2). In the culture systems having a medium conditioned with urothelial bladder stromal cells (culture group e), the number of microvascular bladder endothelial cells and dermal microvascular endothelial cells was enhanced to the 3.2 and 2.9fold respectively, compared to the control group a (FIG. 2).

The use of conditioned media (culture groups b, c, and e) resulted in a penetration of the microvascular bladder endothelial cells inside the matrices, with penetration depth of up to 2.3 mm. In these conditioned systems the formation of cords (tubes) (FIG. 4a) and after 10 to 14 days the formation of capillary networks was also induced. The degree of network formation was dependent on the duration of culture until day 28. Consistent with capillary-like differentiation is was found that microvascular bladder endothelial cells and dermal microvascular endothelial cells respectively formed fully developed capillary lumina with each other, lined with monolayer of microvascular endothelial cells (FIGS. 4B, 4C, and 4D) and stained positive for the von Willebrand factor (FIG. 4D).

In the absence of a conditioned medium (control group a) and in culture systems conditioned with urothelial cells only (control group d) microvascular bladder endothelial cells and dermal microvascular endothelial cells showed minimal penetration into the matrix and formed only a few immature disconnected cords over the culture period.

For evaluation of angiogenesis activity the capillary length in each three histological longitudinal sections of three different cell constructs obtained from each one urinary bladder was determined. Quantitative assessment of three-dimensional in vitro angiogenesis was performed by microscopic measurement of the length of formed tubes in three fields of vision. The results are shown in FIG. 3. Dermal microvascular endothelial cells were the longest under urothelial stromal induction (group e).

In these culture systems microvascular bladder endothelial cells reached an overall capillary length of 240 μm and the dermal microvascular bladder endothelial cells reached an overall capillary length of 2100 μm (FIG. 3).

In the presence of stromal cytokines or urothelial stromal cytokines, not all microvascular bladder endothelial cells and dermal microvascular endothelial cells respectively appeared competent for invasion. The residual attached microvascular bladder endothelial cells and dermal microvascular endothelial cells respectively did not penetrate into the matrix but migrated only on the surface of the matrix and formed multilayers of cells thereon, they thus likely represented a subpopulation of a heterogeneous cell population.

Although urothelial cells are a source of VEGF production, there could not been found an inducing effect of the medium conditioned only with urothelial cells on microvascular endothelial cells. These results suggest that urothelial cells require the additional effect of bladder stromal cells to induce vascularization in vitro. The synergistic effect of urothelial cells and stromal cells showed the highest inducing effect on microvascular endothelial cells with respect to cell proliferation and tube/capillary formation.

### Literature

Alberti C, Tizzani A, Piovano M, Greco A. What's in the pipeline about bladder econstructive surgery? Some remarks on the state of the art Int J Artif Organs. 2004; 27(9): 737-43.
Atala A. New methods of bladder augmentation. BJU Int. 2000; 85Suppl3: 2434; discussion36.
Blau, H. M., and Banfi, A. The well-tempered vessel. Nature Med. 2001; 7, 532-534
Berthod F, Germain L, Tremblay N, Auger F A. J Cell Physiol. Extracellular matrix deposition by fibroblasts is necessary to promote capillary-like tube formation in vitro. 2006 Feb. 1.
Carmeliet, P., and Jam, R. K. Angiogenesis in cancer and other diseases. Nature (London). 2000; 407, 249-257
Darland D C, D'Amore P A. TGF beta is required for the formation of capillary-like structures in three-dimensional cocultures of 10T112 and endothelial cells. Angiogenesis. 2001 4(1): 11-20.
Erdag G, Sheridan R L. Fibroblasts improve performance of cultured composite skin substitutes on athymic mice. Burns. 2004; 30(4): 322-8.
Ferrara, N., and Alitalo, K. Clinical applications of angiogenic growth factors and their inhibitors. Nature Med. 1999; 5, 1359-1364.
Gruber R, Kandler B, Holzmann P, Vogele-Kadletz M, Losert U, Fischer M B, Watzek G. Bone marrow stromal cells can provide a local environment that favors migration and formation of tubular structures of endothelial cells. Tissue Eng. 2005; 11 (5-6): 896-903.
Honorati M C, Neri S, Cattini L, Facchini A. Interleukin-17, a regulator of angiogenic factor release by synovial fibroblasts. Osteoarthritis artilage. 2005; 23; [Epub ahead of print].
Hudon V, Berthod F, Black AF, Damour 0, Germain L, Auger F A. A tissue-engineered endothelialized dermis to study the modulation of angiogenic and angiostatic molecules on capillary-like tube formation in vitro. Br J Dermatol. 2003; 148(6): 1094-104.
Jam R K, Au P, Tam J, Duda D G, Fukumura D. Engineering vascularized tissue. Nat Biotechnol. 2005; 23(7): 821-3.
Keshet, E., and Ben-Sasson, S. A. Anticancer drug targets: approaching angiogenesis. J. Clin. Invest. 1999; 104, 1497-1501.
Lazarous D F, Shou M, Stiber J A, Dadhania D M, Thirumurti V, Hodge E, Unger E F. Pharmacodynamics of basic fibroblast growth factor: route of administration determines myocardial and systemic distribution. Cardiovasc Res. 1997; 36(1): 78-85.
Markowicz*, E. Koellensperger, S. Neuss, G. C. M. Steffens and N. Pallua. Enhancing the Vascularization of Three-Dimensional Scaffolds: New strategies in Tissue Regeneration and Tissue Engineering. Topics in Tissue Engineering, Volume 2, 2005.
Mertsching H, Wailes T, Hofmann M, Schanz J, Knapp W H. Engineering of a vascularized scaffold for artificial tissue and organ generation. Biomaterials. 2005; 26(33): 6610-7.
Mooney D J, Mikos A G. Growing new organs. Sci Am. 1999 April; 280(4): 60-5.
Omaida C. Velazquez, Ruthanne Snyder, Zhao-Jun Liu, Ronald M. Fairman., and Meenhard Herlyn Fibroblast-dependent differentiation of human microvascular endothelial cells into capillary-like, three-dimensional networks. The FASEB Journal express article 10.1096/fj.01-IOIIfje. Published online Jun. 7, 2002.
Pepper M S, Ferrara N, Orci L, Montesano R. Potent synergism between vascular endothelial growth factor and basic fibroblast growth factor in the induction of angiogenesis in vitro. Biochem Biophys Res Commun. 1992; 189(2): 824-31.
Risau W, Flamme I. Vasculogenesis. Annu Rev Cell Dev Biol. 1995; 11: 73-91.
Tang Y L, Zhao Q, Zhang Y C, Cheng L, Liu M, Shi J, Yang Y Z, Pan C, Ge J, Phillips M I. Autologous mesenchymal stem cell transplantation induce VEGF and neovascularization in ischemic myocardium. Regul Pept 2004; 117: 3-10.
Thompson H. G., Truong D. T., Griffith C. K., George S. C. A three-dimensional in vitro model of angiogensis in the airway mucosa. Pulm Pharmacol Ther. 2006, January 13.
Velazquez O. C., Snyder R., Lin Z., Fairman R. M., Herlyn M. Fibroblast-dependent differentiation of human microvascular endothelial cells into capillary-like, three-dimensional networks. The FASEB Journal 10.1096; 06.2002.

Watanabe M, Fujioka-Kaneko Y, Kobayashi H, Kiniwa M, Kuwano M, Basaki Y. Involvement of integrin-linked kinase in capillary/tube-like network formation of human vascular endothelial cells. Biol Proced Online. 2005; 7: 41-7. Epub 2005 Apr. 27.

Yancopoulos, G. D., Davis, S., Gale, N. W., Rudge, J. S., Wiegand, S. J., Holash, J. Vascular-specific growth factors and blood vessel formation. Nature (London). 2000; 407, 242-248.

1. A tissue transplant construct for the reconstruction of a human or animal organ, comprising
(a) a biocompatible acellular membrane and
(b) microvascular endothelial cells, which penetrate the membrane,
wherein microvascular structures of microvascular endothelial cells are formed inside the membrane.

2. The tissue transplant construct according to claim 1, characterized in that the human or animal organ is selected from the group comprising the urinary bladder, the ureter, and the urethra.

3. The tissue transplant construct according to claim 1 or claim 2, characterized in that the microvascular endothelial cells are microvascular bladder endothelial cells.

4. The tissue transplant construct according to claim 1 or claim 2, characterized in that the microvascular endothelial cells are dermal microvascular endothelial cells.

5. The tissue transplant construct according to any one of the preceding claims, characterized in that the microvascular endothelial cells are organ-specific microvascular endothelial cells.

6. The tissue transplant construct according to any one of the preceding claims, characterized in that the microvascular endothelial cells are autologous microvascular endothelial cells.

7. The tissue transplant construct according to any one of the preceding claims, characterized in that the microvascular structures comprise lumina.

8. The tissue transplant construct according to any one of the preceding claims, characterized in that the microvascular structures are cross-linked.

9. The tissue transplant construct according to any one of the preceding claims, characterized in that the microvascular structures have been developed in vitro.

10. The tissue transplant construct according to any one of the preceding claims, characterized in that the membrane is penetrated by further tissue-specific cells.

11. A method for the preparation of a tissue transplant construct for the reconstruction of a human or animal organ, comprising the steps of
(a) isolating microvascular endothelial cells;
(b) applying the microvascular endothelial cells onto a biocompatible acellular membrane, and
(c) cultivating the microvascular endothelial cells, which have been applied onto the biocompatible acellular membrane, under stromal induction or under epithelial-stromal induction.

12. The method according to claim 11, characterized in that the human or animal organ is selected from the group comprising the urinary bladder, the ureter, and the urethra.

13. The method according to claim 11 or claim 12, characterized in that the microvascular endothelial cells are microvascular bladder endothelial cells.

14. The method according to claim 11 or claim 12, characterized in that the microvascular endothelial cells are dermal microvascular endothelial cells.

15. The method according to any one of claims 11 to 14, characterized in that the microvascular endothelial cells are organ-specific microvascular endothelial cells.

16. The method according to any one of claims 11 to 15, characterized in that the microvascular endothelial cells are autologous microvascular endothelial cells.

17. The method according to any one of claims 11 to 16, characterized in that the stromal induction is performed by using human or animal bladder stromal cells or human or animal marrow stromal progenitor cells.

18. The method according to any one of claims 11 to 17, characterized in that the culturing under stromal induction is performed by means of a conditioned medium, wherein the conditioned medium has been conditioned by means of human or animal bladder stromal cells or human or animal marrow stromal progenitor cells.

19. The method according to claim 18, characterized in that the conditioned medium is obtained by cultivating non-conditioned medium with human or animal bladder stromal cells or human or animal marrow stromal progenitor cells and subsequently removing it as supernatant from the bladder stromal cells or marrow stromal progenitor cells.

20. The method according to any one of claims 11 to 16, characterized in that the epithelial-stromal induction is performed by using human or animal urothelial bladder stromal cells.

21. The method according to claim 20, characterized in that the culturing under epithelial-stromal induction is performed by means of a conditioned medium, wherein the conditioned medium has been conditioned by means of human or animal urothelial bladder stromal cells.

22. The method according to claim 21, characterized in that the conditioned medium is obtained by culturing a non-conditioned medium with human or animal urothelial bladder stromal cells and subsequently removing it as a supernatant from the urothelial bladder stromal cells.

23. The method according to any one of claims 11 to 22, characterized in that the microvascular endothelial cells are recovered from the stromal tissue of the organ to be reconstructed.

24. The method according to any one of claims 14 to 22, characterized in that the dermal microvascular endothelial cells are recovered from the dermis.

25. The method according to claim 23, characterized in that the microvascular endothelial cells are recovered from the stromal tissue of the organ to be reconstructed by
(i) digesting the stromal tissue by means of a collagenase;
(ii) separating the microvascular endothelial cells from the mixture obtained in step (i) using paramagnetic lectine- or antibody-coupled particles, wherein the antibodies are monoclonal antibodies for the platelet-endothelial cell adhesion molecule 1 (PECAM 1);
(iii) propagating the thus obtained microvascular endothelial cells; and
(iv) separating the microvascular endothelial cells from the mixture obtained in step (iii) using paramagnetic lectine- or antibody-coupled particles, wherein the antibodies are monoclonal antibodies for the platelet-endothelial cell adhesion molecule 1 (PECAM 1) and wherein the thus obtained microvascular endothelial cells are a mixture with a purity of at least 95% based on the number of all cells.

26. The method according to claim 25, characterized in that the residue of the mixture remained in step (ii) is used to prepare the conditioned medium.

27. The method according to any one of claims 11 to 26, characterized in that after completion of culturing the microvascular endothelial cells, which have been applied onto the biocompatible acellular membrane, under stromal induction or under urothelial-stromal induction (step (c)) further tissue-specific cells are applied to the membrane and are cultured thereon.

28. The use of a tissue transplant construct according to any one of claims 1 to 10 for the reconstruction of a human or animal organ.

29. The use according to claim 28, wherein the organ is selected from the group comprising the urinary bladder, the ureter, and the urethra.
